(19) United States
(12) Patent Application Publication
(10) Pub. No.: US 2019/0187135 A1
(43) Pub. Date: Jun. 20, 2019

(54) GAS SENSOR USING METAL NANOPARTICLES

(71) Applicant: ELECTRONICS AND TELECOMMUNICATIONS RESEARCH INSTITUTE, Daejeon (KR)

(72) Inventors: Young Jun KIM, Daejeon (KR); Jae Doo HUH, Daejeon (KR); Bongjin JEONG, Daejeon (KR)

(21) Appl. No.: 16/214,511

(22) Filed: Dec. 10, 2018

(30) Foreign Application Priority Data
Dec. 15, 2017 (KR) 10-2017-0173626

(51) Int. Cl. G01N 33/543; G01N 33/497; G01N 33/553; G01N 27/327

(52) U.S. Cl. CPC G01N 33/5438; G01N 33/497; G01N 27/3278; G01N 33/54346; G01N 33/553

(57) ABSTRACT

A gas sensor includes a substrate, a pair of electrodes formed on the substrate, having a nanogap formed therebetween, and facing each other, and metal nanoparticles present in the nanogap, wherein a ligand organic single-molecule having a gas-bonding functional group and a ligand organic single-molecule having a substrate-bonding functional group are disposed on the surface of the metal nanoparticles, and wherein an organic single-molecule having a substrate-functional group bonding to the substrate-bonding functional group of the ligand organic single-molecule having a substrate-bonding functional group disposed on the surface of the metal nanoparticles is immobilized to the substrate, a method for manufacturing the gas sensor, and a use thereof.

### CROSS-REFERENCE TO RELATED APPLICATION

This application claims priority to and the benefit of Korean Patent Application No. 10-2017-0173626 filed in the Korean Intellectual Property Office on Dec. 15, 2017, the entire content of which is incorporated herein by reference.

### BACKGROUND OF THE INVENTION

### 1. Field of the Invention

The present invention relates to a gas sensor using metal nanoparticles. More particularly, the present invention relates to a gas sensor including metal nanoparticles capable of sensing a gas with an ultra-high sensitivity, wherein the metal nanoparticles includes a ligand organic single-molecule improving gas selectivity.

### 2. Description of Related Art

A metal oxide sensor has been mainly used as a conventional gas sensor. In this case, attempts have been made to sense a variety of materials by mainly adding various catalytic metals to metal oxides to change oxidation-reduction property, which is the basic sensing mechanism of the metal oxide sensor. For example, International Patent Publication WO 2010/126336 A2 discloses a gas sensor including a sensor substrate provided with an electrode and a thin layer of sensor material formed by spraying a solution in which the metal oxide nanoparticles are dispersed onto the sensor substrate. However, the metal oxide sensor has a limitation in expanding of the sensing target through diversification of sensor characteristics using the catalyst and a necessity of electric power depending on the operation thereof at a high temperature, which are the main difficulties in being applied to a sensor array technology, particularly, a miniaturized sensor array technology.

In the sensor array technology, various sensors or sensor technologies have recently been developed to overcome the limitations of the related art. Among others, metal nanoparticles have been recently actively studied as sensor materials capable of overcoming the limitations of the metal oxide sensor.

For example, Korean Patent Laid-Open Publication No. 10-2010-0108983 discloses a thin film gas sensor with high activity using core-shell structure composite nanoparticles composed of a metal nanoparticles core and a metal oxide nanoparticles shell layer surrounding the surface of the core, as a sensing material.

On the other hand, attempts have recently been made to use the sensor array for diagnosing diseases. It has been recognized as a major advantage that a disease diagnosis using this sensor array enables a noninvasive real-time disease diagnosis through analysis of human respiratory gas or secretions. Recently, it has been reported that a gold nanoparticles sensor having a size of about 2 nm with octanethiol as a ligand has a sensitivity of several ppm for toluene. However, it has been reported that this octanethiol-gold nanoparticle has excellent sensitivity for non-polar molecules such as toluene or CCl₄, but very week sensitivity for polar molecules. Attempts have been made to use the ligands composed of alcohol (—OH) or ethylene oxide to improve sensitivity to the polar molecules. However, although the sensitivity has been improved by using such a polar ligand, the reduction in the reaction time and the stabilization in the reaction curve are still required to be achieved.

There is a need for a gas sensor that capable of accurately determining whether a specific disease occurs by analyzing the respiratory gas and accurately selecting a specific gas among them to calculate the concentration thereof.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a gas sensor using metal nanoparticles having high selectivity and reliability, in order to solve the above-mentioned problems of the related art.

In addition, the present invention has been made in an effort to provide a respiratory gas sensor having high selectivity and reliability capable of determining in real time whether a disease occurs.

An exemplary embodiment of the present invention provides,

a gas sensor including a substrate, a pair of electrodes formed on the substrate, having a nanogap formed therebetween, and facing each other, and metal nanoparticles present in the nanogap, wherein a ligand organic single-molecule having a gas-bonding functional group and a ligand organic single-molecule having a substrate-bonding functional group are disposed on the surface of the metal nanoparticles, and wherein an organic single-molecule having a substrate-functional group bonding to the substrate-bonding functional group of the ligand organic single-molecule having a substrate-bonding functional group disposed on the surface of the metal nanoparticles is immobilized to the substrate

The ligand organic single-molecule having the gas-bonding functional group may include a bonding portion binding to the surface of the metal nanoparticles, the gas-bonding functional group, and a connecting portion connecting the bonding portion and the gas-bonding functional group. The bonding portion may be selected from a thiol group (—SH), an amino group (—NH₂), a carboxyl group (—COOH), or a phosphate group (—H₂PO₄), the connecting portion may be selected from a C1 to C10 alkylene group, a C6 to C30 arylene group, or —(CH₂CH₂O)ₙ— (wherein n is 1-10), and the gas-bonding functional group may be selected from —R, —OR, —COOR, —COR, —NR¹R² (wherein R, R¹ and R² are each independently hydrogen, a C1 to C10 alkyl group, a C6 to C30 aryl group, or combinations thereof), and halogen.

The ligand organic single-molecule having the substrate-bonding functional group may include a bonding portion binding to the surface of the metal nanoparticles, the substrate-bonding functional group, and a connecting portion connecting the bonding portion and the substrate-bonding functional group. The bonding portion may be selected from a thiol group (—SH), an amino group (—NH₂), a carboxyl group (—COOH), or a phosphate group (—H₂PO₄), the connecting portion may be selected from a C1 to C10 alkylene group, a C6 to C30 arylene group, or —(CH₂CH₂O)ₙ— (wherein n is 1-10), and the substrate-bonding functional group may be selected from —NR¹R², —OR, —COOR, and —COR (wherein R, R¹ and R² are each independently hydrogen, a C1 to C10 alkyl group, a C6 to C20 aryl group, or combinations thereof).

The organic single-molecule having the substrate-functional group may be selected from a trialkoxysilane derivative.

In the organic single-molecule having the substrate-functional group, the substrate-functional group selected from —NR¹R², —OR, —COOR, or —COR (wherein R, R¹ and R² are each independently —H, —CH₃, —CH₂CH₃ or —C₆H₅) may be bonded to one terminal of trialkoxysilane.

The electrode may have an interdigitate (IDT) electrode structure and the nanogap may have a width in the range of 10 nm-1000 nm.

The metal nanoparticles on which the ligand organic single-molecule having the gas-bonding functional group and the ligand organic single-molecule having the substrate-bonding functional group are disposed may be formed in the nanogap as a monolayer.

In the gas sensor of the present invention, the metal nanoparticles on which the ligand organic single-molecule having the gas-bonding functional group and the ligand organic single-molecule having the substrate-bonding functional group are disposed may be stacked in the nanogap as a plurality of layers.

The metal nanoparticles may include at least one metal selected from Au, Ag, Pt, Pd, Ir, Rh, or any alloys thereof.

The electrode may include at least one selected from gold, silver, platinum, carbon nanotubes, graphene, polypyrrole, polyaniline, polythiophene, or polyethylenedioxythiophene (PEDOT).

The nanoparticles may include at least one of a nanosphere, a nanowire, a nanorod, a nanowall, a nanotube, a nanobelt, and a nanoring.

The substrate may be a silicon substrate or a glass substrate.

According to an exemplary embodiment of the present invention, the gas sensor using the metal nanoparticles according to the present invention may have improved sensitivity to sensed targets and response speed, and have excellent selectivity for various sensed targets, such that the types and compositions of the ligand organic single-molecule constituting the metal nanoparticles sensor may be adjusted, thereby making it possible to provide an ultra-high sensitivity gas sensor.

In addition, the types and the ratio of compositions of the ligand organic single-molecule are adjusted to enable the design of gas sensor characteristics, thereby making it possible to perform a systematic approach to the most efficient sensor array configuration for the analysis targets. The application of the high-sensitivity metal nanoparticle sensor to array technology is expected to significantly contribute to a non-invasive real-time disease diagnosis technology through human respiratory gas or other secretions.

According to the present invention, a respiratory gas sensor for diagnosis having high selectivity and reliability, capable of determining, in real time, whether or not there is an abnormality or whether or not a specific disease occurs may be provided.

### BRIEF DESCRIPTION OF THE DRAWINGS

FIG. 1 is a schematic cross-sectional view showing a gas sensor using metal nanoparticles according to an exemplary embodiment of the present invention.

FIG. 2 a configuration diagram explaining the metal nanoparticles of the gas sensor, used as a sensing material, according to an exemplary embodiment of the present invention.

FIG. 3 is a schematic diagram showing a gas sensor using metal nanoparticles according to an exemplary embodiment of the present invention.

### DETAILED DESCRIPTION OF THE EMBODIMENTS

Hereinafter, preferred embodiments of the present invention will be described below in detail with reference to the accompanying drawings. In order to facilitate entire understanding of the present invention in describing the present invention, the same reference number in the drawings will be used to describe the same components and an overlapped description of the same components will be omitted.

In the present application, an expression used in the singular encompasses the expression of the plural, unless the context explicitly indicated otherwise. In the present application, it should be understood that terms such as “include” or “have” are intended to indicate the presence of features, numbers, steps, operations, components, parts, or combinations thereof disclosed in the specification, and are not intended to preclude the presence or addition possibilities of one or more other features, numerals, steps, operations, components, parts, or combinations thereof.

Unless indicated otherwise, all the terms used herein, including technical or scientific terms, have the same meanings as those generally understood by those with ordinary knowledge in the field of art to which the present invention belongs.

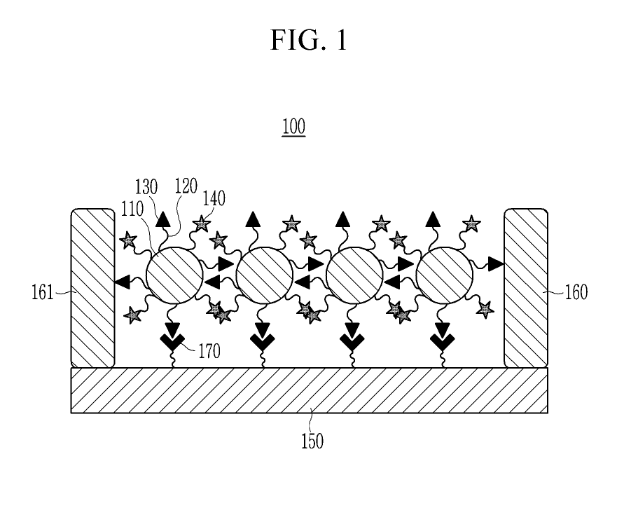

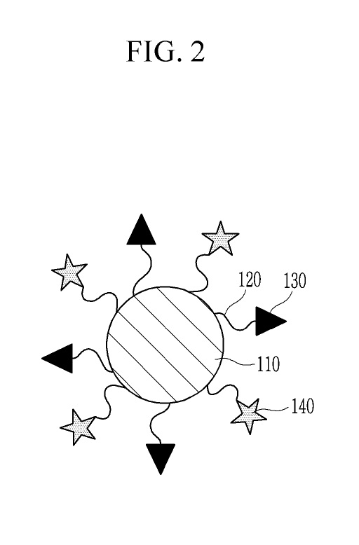

FIG. 1 is a schematic cross-sectional diagram showing a gas sensor using metal nanoparticles according to an exemplary embodiment of the present invention. FIG. 2 is a configuration diagram explaining the metal nanoparticles of the gas sensor, used as a sensing material, according to an exemplary embodiment of the present invention.

Referring to FIGS. 1 and 2, a gas sensor 100 using the metal nanoparticles according to an exemplary embodiment of the present invention includes a substrate 150 provided with electrodes 160 and 161 having a nanogap formed therebetween, and metal nanoparticles 110 present in the nanogap, wherein a ligand organic single-molecule having a gas-bonding functional group 140 bonding to a gas introduced into the nanogap, and a ligand organic single-molecule having a substrate-bonding functional group 130 are disposed on the surface of the metal nanoparticles 110, and wherein an organic single-molecule having a substrate-functional group 170 bonding to the substrate-bonding functional group 130 of the metal nanoparticles is immobilized to the substrate 150. The ligand organic single-molecule having the substrate-bonding functional group 130 and the substrate-functional group 170 may be chemically or physically bonded to each other, and, for example, may be covalently bonded to each other.

Particularly, in the gas sensor 100 using the metal nanoparticles according to an exemplary embodiment of the present invention, the metal nanoparticles on which the ligand organic single-molecule having a gas-bonding functional group 140 and the ligand organic single-molecule having a substrate-bonding functional group 130 are disposed may be formed on the substrate 150 as a monolayer. As described above, the metal nanoparticles are formed on the substrate as a monolayer, such that the resistance may be controlled by changing the size of the metal nanoparticles and the size of the ligand organic single-molecule having the gas-bonding functional group, and at the same time, the resistance may also be controlled by changing the gap between the electrodes from several nanometers to several tens of nanometers. Particularly, in the gas sensor according to the present invention, the gap between the electrodes may be finely formed in a nanometer unit, such as, for example, from several nanometers to several tens of nanometers, such that a minute change in electrical resistance may be detected depending on the presence or absence of a trace amount of gas. As a result, the gas sensor having a significantly increased sensitivity may be provided.

In another exemplary embodiment, the metal nanoparticles in the gas sensor of the present invention may be stacked in the nanogap between the electrodes facing each other as a plurality of layers. When the metal nanoparticles are formed as a plurality of layers, the gas sensor may capture gas molecules three-dimensionally, making it possible to further amplify the gas sensitivity.

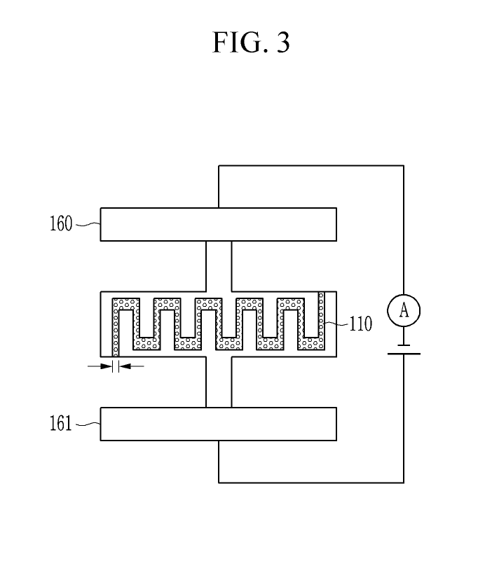

Referring to FIG. 3, in a gas sensor according to an exemplary embodiment, two electrodes 160 and 161 face each other in a comb shape, and a nano-sized gap, that is, a nanogap, is formed between the two electrodes facing each other in a ruggedness portion in the comb shape. The size of the nanogap may be designed to be between 5 nm and 1000 nm, and the smaller the size, the higher the sensitivity of gas-sensing. As shown in the ruggedness shape in FIG. 3, from the two electrodes facing each other, the nanogap having the ruggedness shape may be formed to have at least one pair, for example, at least 10 pairs, for example, at least 100 pairs, for example, at least 200 pairs, and, for example, at least 300 pairs. The nanogap is formed to have a plurality of pairs of ruggedness shapes as described above, whereby the change in electrical resistance depending on sense of gas can be further amplified.

The ligand organic single-molecule having the gas-bonding functional group 140 may include a bonding portion binding to the surface of the metal nanoparticles, a gas-bonding functional group 140, and a connecting portion 120 connecting the bonding portion and the gas-bonding functional group

The bonding portion may be selected from a thiol group (—SH), an amino group (—NH₂), a carboxyl group (—COOH), or a phosphate group (—H₂PO₄), the connecting portion 120 may be selected from a C1 to C10 alkylene group, a C6 to C30 arylene group, or —(CH₂CH₂O)ₙ— (wherein n is 1-10), and the gas-bonding functional group 140 may be selected from —R, —OR, —COOR, —COR, —NR¹R² (wherein R, R¹ and R² are each independently hydrogen, a C1 to C10 alkyl group, a C6 to C30 aryl group, or combinations thereof), or halogen.

In an exemplary embodiment, the connecting portion may be selected from a C1 to C6 alkylene group, a phenylene group, or —(CH₂CH₂O)ₙ— (wherein n′ is 1 to 4), but not limited thereto.

In an exemplary embodiment, the gas-bonding functional group may be selected from a methyl group, an ethyl group, a carboxyl group, a methoxy group, an ethoxy group, an amino group, a phenyl group, a chloro group or a bromo group, but not limited thereto.

In the present invention, the ligand organic single-molecule having the gas-bonding functional group (140) disposed on the surface of the metal nanoparticles may also composed of at least two different ligand organic single-molecules, having selectivity for analytes. At least two different ligand organic single-molecules may be various combinations of molecules that are chemically or structurally different from each other. These combinations may also be formed of combinations of different family of compounds, and also may be formed of combinations of molecules of the same family of compounds but with different numbers of carbon atoms or functional group. When the ligand organic single-molecule having the gas-bonding functional group 140 is composed of at least two different ligand organic single-molecules having selectivity for analytes, not only one gas but also at least two different gases may be simultaneously detected.

The ligand organic single-molecule having the substrate-bonding functional group 130 renders the metal nanoparticles 110 to be bonded to the substrate. The ligand organic single-molecule having the substrate-bonding functional group 130 particularly may be physically or chemically bonded to the substrate-functional group 170 formed on the substrate, and in an exemplary embodiment, the bonding may be a chemical bonding, and thus, the substrate-bonding functional group 130 and the substrate-functional group 170, for example, may be covalently bonded to each other.

The ligand organic single-molecule having the substrate-bonding functional group 130 may include a bonding portion binding to the surface of the metal nanoparticles, the substrate-bonding functional group 130, and a connecting portion 120 connecting the bonding portion and the substrate-bonding functional group 130, wherein the bonding portion may be selected from a thiol group (—SH), an amino group (—NH₂), a carboxyl group (—COOH), or a phosphate group (—H₂PO₄), the connecting portion 120 may be selected from a C1 to C10 alkylene group, a C6 to C30 arylene group, or —(CH₂CH₂O)ₙ— (wherein n is 1-10), and the substrate-bonding functional group 130 may be selected from —NR¹R², —OR, —COOR, or —COR (wherein R, R¹ and R² are each independently hydrogen, a C1 to C10 alkyl group, a C6 to C20 aryl group, or combinations thereof).

In an exemplary embodiment, the connecting portion may be selected from a C1 to C6 alkylene group, a phenylene group, or —(CH₂CH₂O)ₙ— (wherein n′ is 1 to 4), but not limited thereto.

In an exemplary embodiment, the substrate-bonding functional group may be any one of —COOH, —NH₂, and —OH, but not limited thereto.

The organic single-molecule having the substrate-functional group may be selected from a trialkoxysilane derivative. In the organic single-molecule having the substrate-functional group, the substrate-functional group selected from —NR¹R², —OR, —COOR, or —COR (wherein R, R¹ and R² are each independently —H, —CH₃, —CH₂CH₃ or —C₆H₅) may be bonded to one terminal of trialkoxysilane. In an exemplary embodiment, the substrate-functional group may be any one of COOH, —NH₂ and —OH, but not limited thereto.

The metal nanoparticles 110 may be composed of Au, Ag, Pt, Pd, Ir, Rh, or any alloys thereof. In an exemplary embodiment, the metal nanoparticles may be composed of Au nanoparticles. In the present invention, the metal nanoparticles may have a size of 2 nm to 100 nm. The nanoparticles according to the present invention do not necessarily mean only spherical particles, but may also be nanostructures such as a nanowire, a nanorod, a nanowall, a nanotube, a nanobelt, and a nanoring.

The metal nanoparticles on which the ligand organic single-molecule of the present invention is disposed may be synthesized according to a known synthesis method of nanoparticle. For example, it may be prepared according to the methods described in the document [M. Brust, M. Walker, D. Bethell, D. J. Schiffrin and R. Whyman, J. Chem. Soc., Chem. Commun. 1994 801-802]; and the document [Michael J. Hostetler, Stephen J. Green, Jennifer J. Stokes and Royce W. Murray “Monolayers in Three Dimensions: Synthesis and Electrochemistry of omega-Functionalized Alkanethiolate-Stabilized Gold Cluster Compounds” J. Am. Chem. Soc. 1996, 118, 4212-4213].

In the present invention, the electrode may be an interdigitate electrode (IDE) having an IDT structure. As shown in FIG. 3, the IDE electrode is an electrode in which two electrodes having a comb structure face each other in a ruggedness shape, and the gap between the ruggedness portions may be several nanometers to several hundreds or 1,000 nm. The IDE electrode may be obtained by forming an electrode on a substrate such as a conventional slide glass. Specific materials constituting the substrate and the electrode are not particularly limited, and known materials used in the conventional IDE electrode structure may be used. For example, the electrode 160 may be formed of gold, silver, platinum, carbon nanotubes, or graphene, or may be formed of a conductive polymer such as polypyrrole, polyaniline, polythiophene and polyethylenedioxythiophene (PEDOT). The IDE electrode may be formed on the substrate using a lithography method, in particular, an electron beam lithography (e-beam lithography).

In the present invention, the substrate 150 may be made of any one of a silicon and a glass substrate, and the organic single-molecule having the substrate-functional group 170 immobilized to the substrate may include the substrate-functional group 170 and any connection portion capable of immobilizing the substrate-functional group 170 to the substrate 150. The substrate-functional group 170 may be any one selected from —COOR, —COR, —NR¹R², or —OR (wherein R, R¹ and R² are each independently hydrogen, a C1 to C10 alkyl group, a C6 to C20 aryl group, or combination thereof), and the organic single-molecule having the substrate-functional group 170 may be a trialkoxysilane derivative. That is, the organic single-molecule may be selected from the trialkoxysilane derivative having one terminal including a silicon-containing group such as a trimethoxysilyl group and a triethoxysilyl group, capable of being bonded to a silicon or glass substrate, the other end including the substrate functional group 170, and a connecting portion, such as, a C1 to C10 alkylene group, between the silicon-containing group and the substrate functional group 170.

The gas sensor using metal nanoparticles according to an exemplary embodiment of the present invention has excellent detection sensitivity for a specific gas and a fast response speed. In addition, not only the ligand organic single-molecule having the gas-bonding functional group 140 but also the ligand organic single-molecule having the substrate-bonding functional group 130 bonded to the substrate-functional group 170 are bonded to the surface of the metal nanoparticles, such that the metal nanoparticles 110 may be stably bonded and immobilized to the substrate 150. Accordingly, the metal nanoparticles 110 of the gas sensor according to the present invention do not fall off from the nanogap despite being used for a long time, such that the durability of the gas sensor is maintained. As a result, the electrical characteristics of the gas sensor is not deteriorated or changed, whereby the gas sensor has excellent reliability.

A gas sensor including metal nanoparticles according to an exemplary embodiment of the present invention may be manufactured by forming electrodes having a nanogap on a substrate; introducing and binding an organic single-molecule having a substrate functional group 170 into the nanogap; introducing metal nanoparticles 110 to the organic single-molecule having a substrate functional group 170, introducing metal nanoparticles 110 to which a ligand organic single-molecule having a gas-bonding functional group 140 and a ligand organic single-molecule having a substrate-bonding functional group 130 are disposed, and thereby binding the substrate functional group 170 formed on the substrate to the substrate-bonding functional group 130 of the metal nanoparticles to each other.

The method of disposing the ligand organic single-molecule having the gas-bonding functional group and the ligand organic single-molecule having the substrate-bonding functional group on the surface of the metal nanoparticles to be immobilized may include any of the following methods: a two-phase bonding method in which the metal nanoparticles and the ligand organic single-molecules having each of functional group are sequentially bonded to each other; or a ligand exchange reaction in which only the ligand organic single-molecule having any one of the two functional groups is bonded and then some of the functional groups are replaced with another functional group.

The electrode having an IDT structure may be formed on the substrate using a lithography method, in particular, an e-beam lithography method.

The method of bonding the substrate-bonding functional group 130 to the substrate functional group 170 is not limited to a specific method. However, in an exemplary embodiment, the substrate-bonding functional group 130 and the substrate functional group 170 may be bonded to each other through a covalent bond. In this case, the substrate-bonding functional group 130 and the substrate functional group 170 may be selected from any functional group capable of being covalently bonded with each other. As the substrate-bonding functional group 130 and the substrate functional group 170 are bonded to each other, the metal nanoparticles having the surfaces thereof on which the ligand organic single-molecule having the substrate-bonding functional group is disposed may be stably immobilized to the substrate, and these metal nanoparticles may be formed on the substrate as a monolayer.

The gas sensor using the metal nanoparticles of the present invention may be manufactured by forming a film on a substrate using a polymer solution in which the metal nanoparticles, on which the ligand organic single-molecules are dispose, are dispersed, through a spin coating method, a dip-coating method, a dispensing method, and various other known methods.

The gas sensor of the present invention may further include a current or resistance measuring unit. The current or resistance measuring unit measures the current or resistance value of the metal nanoparticles 110 in the nanogap of a sensing part composed of the metal nanoparticles 110 on which the ligand organic single-molecule having the gas-bonding functional group and the ligand organic single-molecule having the substrate-bonding functional group are disposed. When the gas to be detected is introduced into the nanogap between the electrodes of the gas sensor of the present invention, a change occurs in the electrical resistance value between the electrodes by an interaction between the gas and the combined body of the metal nanoparticles and the ligand organic single-molecule having the gas-bonding functional group, and in this case, the change in the resistance value is detected, thereby making it possible to determine the presence of the specific gas or to measure the concentration of the specific gas. Specifically, as the gas, such as a respiratory gas is injected into the inside of the nanogap of the gas sensor and is thus adsorbed into the gas-bonding functional group of the ligand organic single-molecule having the gas-bonding functional group disposed on the surface of the metal nanoparticles 110, a length of the ligand organic single-molecule is lengthened due to the adsorption of the gas so that a distance between the metal nanoparticles becomes longer, resulting in that the electrical resistance value in the nanogap varies. From this, it is possible to determine the presence of the specific gas and to calculate the concentration of the specific gas. The resistance measuring unit may be constructed according to the related art, and there is no need to be particularly limited to the configuration of the resistance measuring unit, as long as the electrical resistance of the metal nanoparticles 110 may be accurately measured.

There are hundreds of volatile organic compounds and volatile sulfur compounds gases released through exhaled breath of humans or animals. Among others, certain gases are known to be used as biomarkers including health information of humans or animals. Examples of the volatile organic compounds gases that may be used as the biomarker include acetone, toluene, ammonia, carbon monoxide, and carbon dioxide, etc. Examples of the volatile sulfur compounds gases include hydrogen sulfide, dimethyl sulfide, and methyl mercaptan, etc. Among them, as representative examples, acetone and ammonia gas are known as the biomarkers for diabetes and kidney diseases, respectively, and hydrogen sulfide, dimethyl sulfide and methyl mercaptan gases are known as the biomarkers for halitosis diseases.

The gas sensor of the present invention may simply and accurately detect specific volatile organic compounds in the exhaled breath, and thus may be implemented as an ultra-sensitive respiratory gas sensor capable of monitoring various diseases in a non-invasive manner. Further, the present invention also may be implemented as a diagnostic device capable of not only diagnosing diseases but also measuring the degree of obesity of a human body and checking a degree of decomposition of human body fat to accurately measure the amount of daily exercise, regarding health care.

Hereinafter, the present invention will be described in more detail through Examples. These Examples are only intended to illustrate the present invention, and should not be interpreted as limiting the scope of the present invention by these Examples.

### EXAMPLES

### Preparation Example 1

### Preparation of Gold Nanoparticle to Which Ligand Organic Single-Molecule Having Gas-Bonding Functional Group is Bonded

Tetraoctylammonium bromide 20.0 g was dissolved in 800 ml of toluene, this solution was mixed with a solution of HAuCl₄ (3.5 g, 8.9 mol) dissolved in 300 ml of water, and stirred for several minutes. 4-Methylbenzenethil (1.08 g, 8.7 mol) and sodium borohydride (3.8 g) dissolved in water (250 ml) were added to the mixed solution, followed by stirring for 3 hours, and an organic layer was separated. The organic solvent was evaporated, suspended in ethanol and placed in a refrigerator to induce precipitation. The precipitated product was washed with ethanol and dried to obtain 4-methylbenzenethiol gold nanoparticles (4-MB—AuNP).

### Preparation Example 2

### Preparation of Gold Nanoparticle to Which Ligand Organic Single-Molecule Having Gas-Bonding Functional Group is Bonded

Tetraoctylammonium bromide was dissolved in toluene, this solution was mixed with a solution of HAuCl₄ dissolved in 300 ml of water, and stirred for several minutes. Diaminobenzene and sodium borohydride dissolved in water were added to the mixed solution, followed by stirring for 3 hours, and an organic layer was separated. The organic solvent was evaporated, suspended in ethanol and placed in a refrigerator to induce precipitation. The precipitated product was washed with ethanol and dried to obtain diaminobenzene gold nanoparticles.

### Preparation Example 3

### Preparation of Gold Nanoparticle to Which Ligand Organic Single-Molecule Having Gas-Bonding Functional Group is Bonded

Tetraoctylammonium bromide was dissolved in toluene, this solution was mixed with a solution of HAuCl₄ dissolved in 300 ml of water, and stirred for several minutes. N-butylthiol and sodium borohydride dissolved in water were added to the mixed solution, followed by stirring for 3 hours, and an organic layer was separated. The organic solvent was evaporated, suspended in ethanol and placed in a refrigerator to induce precipitation. The precipitated product was washed with ethanol and dried to obtain n-butylthiol gold nanoparticles.

### Preparation Example 4

### Preparation of Metal Nanoparticle to Which Ligand Organic Single-Molecule Having Gas-Bonding Functional Group is Bonded

Tetraoctylammonium bromide was dissolved in toluene, this solution was mixed with a solution of HAuCl₄ dissolved in 300 ml of water, and stirred for several minutes. SH(CH₂CH₂O)₂—HS and sodium borohydride dissolved in water were added to the mixed solution, followed by stirring for 3 hours, and an organic layer was separated. The organic solvent was evaporated, suspended in ethanol and placed in a refrigerator to induce precipitation. The precipitated product was washed with ethanol and dried to obtain the gold nanoparticles having SH(CH₂CH₂O)₂—HS boned thereto.

### Preparation Example 5

### Production of Metal Nanoparticle to Which Ligand Organic Single-Molecule Having Gas-Bonding Functional Group is Bonded and Ligand Organic Single-Molecule Having Substrate Bonding Functional Group is Additionally Bonded

The 4-methylbenzenethiol gold nanoparticles (4-MB—AuNP, 100 mg) prepared in Preparation Example 1 were mixed with a large amount of 4-hydroxybenzenethiol (14 mg), dissolved in a solvent, and stirred for 5 days. The final product was suspended in a solvent, filtered, and washed with a solvent, and the solvent was evaporated under reduced pressure to obtain the gold nanoparticle to which the ligand organic single-molecule having the gas-bonding functional group was bonded, and 4-hydroxybenzenethiol, which is the ligand organic single-molecule having the substrate bonding functional group, was additionally bonded.

### Preparation Example 6

### Production of Metal Nanoparticle to Which Ligand Organic Single-Molecule Having Gas-Bonding Functional Group is Bonded and Ligand Organic Single-Molecule Having Substrate Bonding Functional Group is Additionally Bonded

The 4-methylbenzenethiol gold nanoparticles (4-MB—AuNP, 100 mg) prepared in Preparation Example 1 were mixed with a large amount of 4-aminobenzenethiol, dissolved in a solvent, and stirred for 5 days. The final product was suspended in a solvent, filtered, and washed with a solvent, and the solvent was evaporated under reduced pressure to obtain the gold nanoparticle to which the ligand organic single-molecule having the gas-bonding functional group was bonded, and 4-aminobenzenethiol, which is the ligand organic single-molecule having the substrate bonding functional group, was additionally bonded.

### Preparation Example 7

### Production of Metal Nanoparticle to Which Ligand Organic Single-Molecule Having Gas-bonding Functional Group is Bonded and Ligand Organic Single-Molecule Having Substrate Bonding Functional Group is Additionally Bonded

The 4-methylbenzenethiol gold nanoparticles (4-MB—AuNP, 100 mg) prepared in Preparation Example 1 were mixed with a large amount of carboxybenzenethiol, dissolved in a solvent, and stirred for 5 days. The final product was suspended in a solvent, filtered, and washed with a solvent, and the solvent was evaporated under reduced pressure to obtain the gold nanoparticle to which the ligand organic single-molecule having the gas-bonding functional group was bonded, and carboxybenzenethiol, which is the ligand organic single-molecule having the substrate bonding functional group, was additionally bonded.

### Preparation Example 8

### Preparation of Gold Nanoparticle to Which Two Types of Ligand Organic Single-Molecules Having Gas-Bonding Functional Group are Bonded

The 4-methylbenzenethiol gold nanoparticles (4-MB—AuNP, 100 mg) prepared in Preparation Example 1 were mixed with a solvent containing bromobenzene thiol and chlorobenzene thiol, and dissolved, followed by stirring for 5 days.

The final product was suspended in a solvent, filtered, and washed with a solvent, and the solvent was evaporated under reduced pressure to obtain the gold nanoparticle to which 4-methylbenzene thiol was bonded as the ligand organic single-molecule having the gas-bonding functional group, and bromobenzenethiol or chlorobenzenethiol, which is the ligand organic single-molecule having respective gas-bonding functional group, was additionally bonded

### Preparation Example 9

### Formation of Electrode Having Interdigitate (IDT) Structure

The substrate 150 formed of any one of silicon and glass was washed, and was spin-coated with photoresist liquid. An IDT electrode array was aligned with the substrate using a stepper or a contact aligner, then exposed, and developed using a green mask to form a photoresist pattern. A metal film was deposited on the substrate on which the photoresist pattern is formed, using any one of E-beam or sputtering, and the photoresist was removed, thereby completing the substrate on which the IDT electrode is formed. In this case, the photoresist pattern is formed so that the nanogap having a desired size is formed between the two electrodes, thereby making it possible to manufacture the IDT electrode having the nanogap formed between the two electrodes.

Meanwhile, in order to attach the substrate-functional group capable of binding to the substrate-bonding functional group bonded to the metal nanoparticles prepared according to Preparation Examples 1 to 8 to the formed nanogap, 3-aminopropyltriethoxysilane (3-APS) solution is coated.

### Preparation Example 10

### Manufacture and Evaluation of Gas Sensor

Before loading the gold nanoparticles prepared in Preparation Examples 5 to 8 above into the nanogap between the IDT electrodes manufactured in Preparation Example 9, the electrode was immersed in a piranha solution (H₂SO₄/H₂O₂=3:1), then sonicated for 1 minute, and washed with methanol and acetone. The gold nanoparticles prepared in the Preparation Examples above were dissolved in a hydrocarbon solvent or a polar solvent, and then the electrode was dip-coated in this solution, thereby obtaining the gas sensor of the present invention in which the nanoparticles were filled between the nanogaps of the electrodes. The gas sensor obtained above may be connected to the current or resistance measuring unit as shown in FIG. 3, thereby making it possible to obtain a gas sensor according to an exemplary embodiment.

The obtained gas sensor is exposed to various kinds of gases, whereby the kind and/or concentration of the gas may be measured by the value obtained from the current or resistance measuring unit of the gas sensor.

The foregoing description is merely illustrative of the technical spirit of the present invention. It will be appreciated by those skilled in the art that various modifications and alterations can be made without departing from the essential characteristics of the present invention. The true scope of protection of the present invention must be defined by the appended claims and it should be construed that all spirits within a scope equivalent thereto are included in the scope of the present invention.

What is claimed is:

1. A gas sensor comprising:
a substrate;
a pair of electrodes formed on the substrate, having a nanogap formed therebetween, and facing each other; and
metal nanoparticles present in the nanogap,
wherein a ligand organic single-molecule having a gas-bonding functional group and a ligand organic single-molecule having a substrate-bonding functional group are disposed on the surface of the metal nanoparticles, and
wherein an organic single-molecule having a substrate-functional group bonding to the substrate-bonding functional group of the ligand organic single-molecule having a substrate-bonding functional group disposed on the metal nanoparticles is immobilized to the substrate.

2. The gas sensor of claim 1, wherein:
the ligand organic single-molecule having the gas-bonding functional group includes a bonding portion binding to the surface of the metal nanoparticles, the gas-bonding functional group, and a connecting portion connecting the bonding portion and the gas-bonding functional group, the bonding portion is selected from a thiol group (—SH), an amino group (—NH₂), a carboxyl group (—COOH), or a phosphate group (—H₂PO₄), the connecting portion is selected from a C1 to C10 alkylene group, a C6 to C30 arylene group, or —(CH₂CH₂O)ₙ— (wherein n is 1-10), and
the gas-bonding functional group is selected from —R, —OR, —COOR, —COR, —NR¹R²(wherein R, R¹ and R² are each independently hydrogen, a C1 to C10 alkyl group, a C6 to C30 aryl group, or combinations thereof), or halogen.

3. The gas sensor of claim 2, wherein the connecting portion is selected from a C1 to C6 alkylene group, a phenylene group, or —(CH₂CH₂O)ₙ— (wherein n′ is 1 to 4).

4. The gas sensor of claim 2, wherein the gas-bonding functional group is selected from a methyl group, an ethyl group, a carboxyl group, a methoxy group, an ethoxy group, an amino group, a phenyl group, a chloro group or a bromo group.

5. The gas sensor of claim 1, wherein:
the ligand organic single-molecule having the substrate-bonding functional group includes a bonding portion binding to the surface of the metal nanoparticles, the substrate-bonding functional group, and a connecting portion connecting the bonding portion and the substrate-bonding functional group, the bonding portion is selected from a thiol group (—SH), an amino group (—NH₂), a carboxyl group (—COOH), or a phosphate group (—H₂PO₄), the connecting portion is selected from a C1 to C10 alkylene group, a C6 to C30 arylene group, or —(CH₂CH₂O)ₙ— (wherein n is 1-10), and the substrate-bonding functional group is selected from —NR¹R², —OR, —COOR, and —COR (wherein R, R¹ and R² are each independently hydrogen, a C1 to C10 alkyl group, a C6 to C20 aryl group, or combinations thereof).

6. The gas sensor of claim 5, wherein the connecting portion is selected from a C1 to C6 alkylene group, a phenylene group, or —(CH₂CH₂O)ₙ— (wherein n′ is 1 to 4).

7. The gas sensor of claim 1, wherein the substrate-bonding functional group is any one of —COOH, —NH₂, and —OH.

8. The gas sensor of claim 1, wherein the ligand organic single-molecule having the gas-bonding functional group includes at least two different the ligand organic single-molecules having a different type of the gas-bonding functional group.

9. The gas sensor of claim 1, wherein the organic single-molecule having the substrate-functional group is selected from a trialkoxysilane derivative.

10. The gas sensor of claim 9, wherein in the organic single-molecule having the substrate-functional group, the substrate-functional group selected from the group consisting of —NR¹R², —OR, —COOR, and —COR (wherein R, R¹ and R² are each independently —H, —CH₃, —CH₂CH₃ or —C₆H₅) is bonded to one terminal of trialkoxysilane.

11. The gas sensor of claim 10, wherein the substrate-functional group is any one of —COOH, —NH₂, and —OH.

12. The gas sensor of claim 1, wherein the electrode has an interdigitate electrode (IDE) structure.

13. The gas sensor of claim 1, wherein the metal nanoparticles are formed in the nanogap as a monolayer.

14. The gas sensor of claim 1, wherein the metal nanoparticles are stacked in the nanogap as a plurality of layers.

15. The gas sensor of claim 1, wherein the metal nanoparticles include at least one of metal selected from Au, Ag, Pt, Pd, Ir, Rh, or two or more alloys thereof.

16. The gas sensor of claim 15, wherein the metal nanoparticles are Au nanoparticles.

17. The gas sensor of claim 1, wherein a width of the nanogap is in the range of 5 nm to 1000 nm.

18. The gas sensor of claim 1, wherein the electrode includes at least one selected from gold, silver, platinum, carbon nanotubes, graphene, polypyrrole, polyaniline, polythiophene, or polyethylenedioxythiophene (PEDOT).

19. The gas sensor of claim 1, wherein the nanoparticles have a nanosphere, a nanowire, a nanorod, a nanowall, a nanotube, a nanobelt, or a nanoring structure.

20. The gas sensor of claim 1, wherein the substrate is a silicon substrate or a glass substrate.
